(19) United States
(12) Patent Application Publication
(10) Pub. No.: US 2024/0408292 A1
(43) Pub. Date: Dec. 12, 2024

(54) DEVICES, SYSTEMS, AND METHODS FOR TISSUE PROCESSING

(71) Applicant: LifeCell Corporation, North Chicago, IL (US)

(72) Inventors: Brian Hamstrom, Mission Viejo, CA (US); Erin M. Black, Bridgewater, NJ (US); Laszlo Romoda, Mission Viejo, CA (US); Nimesh Kabaria, Hillsborough, NJ (US)

(21) Appl. No.: 18/633,972

(22) Filed: Apr. 12, 2024

Related U.S. Application Data
(63) Continuation of application No. 16/710,671, filed on Dec. 11, 2019, now abandoned.
(60) Provisional application No. 62/778,017, filed on Dec. 11, 2018.

(51) Int. Cl. A61M 1/00; B01D 29/23; B01D 29/64; B01L 3/00; G01N 1/28; G01N 1/38

(52) U.S. Cl. CPC A61M 1/88; A61M 1/782; A61M 1/892; B01D 29/23; B01D 29/6476; B01L 3/502; G01N 1/286; G01N 1/38; B01D 2201/16; B01L 2300/042; B01L 2300/06; B01L 2300/0681; B01L 2300/123

(57) ABSTRACT

The present disclosure provides devices and methods for processing and harvesting adipose tissue. The present disclosure provides improved devices, systems, and methods for harvesting, processing, and filtering adipose tissue for autologous fat transfer procedures. The features of the devices and systems of the present disclosure increase efficiency and sterility of adipose tissue processing by reducing the need for users to interrupt the procedure to adjust some part of the system, such as unclogging filter pores or adjusting tubing.

This application is a continuation of U.S. patent application Ser. No. 16/710,671, filed on Dec. 11, 2019, which claims priority under 35 USC § 119 to U.S. Provisional Application 62/778,017 filed on Dec. 11, 2018. The entire contents of these applications are incorporated herein by reference in their entirety.

The present disclosure relates to devices, systems, and methods for processing tissue, and more particularly to processing and harvesting adipose tissue.

Autologous fat transfer is a procedure that involves harvesting adipose tissue from one region of a patient's body (e.g., by liposuction), processing the harvested tissue, and implanting the processed tissue into another region of the patient's body. Autologous fat transfer has numerous clinical applications such as facial contouring, breast reconstruction and/or augmentation, buttock augmentation, and other aesthetic or reconstructive procedures. In addition, autologous fat grafting has been found to have relatively low donor-site morbidity compared with other surgical options.

While existing devices for processing adipose tissue are effective, features that improve the functionality of the device may be beneficial. For example, the fluid fill level of existing devices must be monitored so that the device does not overflow. A stop mechanism can be added so that the fluid level of the device never exceeds its maximum. Also, users may benefit if the outlet port (i.e. extraction port) of the tissue processing device was flexible and configured such that its distal opening did not leak. An additional benefit of this feature is that users do not have to manipulate the device during tissue extraction or when changing syringes, which prevents fat leakage from the extraction port. Further, the filter and blade shape of existing devices can be improved to minimize clogging of the filter pores while maximizing the tissue processing volume of the device. As such, an improved tissue processing device with the features described above would be beneficial for use in autologous fat transfer procedures.

Accordingly, the present disclosure provides systems, devices, and methods for processing and harvesting adipose tissue. Particularly, the disclosed devices, systems, and methods offer an improved means for harvesting and processing adipose tissue.

### SUMMARY

The present disclosure provides devices, systems, and methods for improved tissue processing. The devices, systems, and methods may be used to harvest and process adipose tissue for reinjection into the patient's body.

In an embodiment of the present disclosure, a tissue processing system is provided. The tissue processing system comprises a container. The container includes an exterior wall surrounding an interior volume for holding tissue, a filter structure for processing tissue, a flow management device, and a flexible outlet having a proximal and a distal end. The container further includes a stop mechanism to prevent filling of the container above a maximum fill level. According to some embodiments of the present disclosure, the stop mechanism comprises a valve.

In an embodiment of the present disclosure, a tissue processing system includes a flow management device. The flow management device comprises a first plate having a plurality of first openings passing therethrough. The flow management device also includes a second plate having a plurality of second openings passing therethrough. The flow management device further includes a third plate having one or more third openings passing therethrough. The first plate, second plate, and third plate are operably connected. Setting the third plate in a first position places a first subset of the plurality of first openings in fluid communication with a first subset of the plurality of second opening. Setting the third plate in a second position places a second subset of the plurality of first openings in fluid communication with a second subset of the plurality of second openings. Setting the third plate to a third position places a third subset of the plurality of first openings in fluid communication with a third subset of the plurality of second openings.

### BRIEF DESCRIPTION OF THE DRAWINGS

The embodiments of the present disclosure are illustrated by way of example, and not limitation, in the accompanying figures, wherein:

FIG. 1 illustrates a perspective view of a tissue processing system according to various embodiments of the present disclosure.

FIG. 2 illustrates a side view of the tissue processing system from FIG. 1, according to various embodiments of the present disclosure.

FIG. 3 illustrates a first cross-sectional view of the tissue processing system from FIG. 1, according to various embodiments of the present disclosure.

FIG. 4 illustrates a second cross-sectional view of the tissue processing system from FIG. 1 in a vertical plane normal to the cross-sectional plane from FIG. 3, according to various embodiments of the present disclosure.

FIG. 5 illustrates an exploded and assembled view of a filter structure according to various embodiments of the present disclosure.

FIG. 6 illustrates a perspective view of an alternative embodiment of the tissue processing system.

### DESCRIPTION OF CERTAIN EXEMPLARY EMBODIMENTS

Reference will now be made in detail to certain exemplary embodiments according to the present disclosure, certain examples of which are illustrated in the accompanying drawings. Wherever possible, the same reference numbers will be used throughout the drawings to refer to the same or like parts.

In this application, the use of the singular includes the plural unless specifically stated otherwise. In this application, the use of “or” means “and/or” unless stated otherwise. Furthermore, the use of the term “including,” as well as other forms such as “included” and “includes,” is not limiting.

The section headings used herein are for organizational purposes only and are not to be construed as limiting the subject matter described. All documents, or portions of documents, cited in this application including but not limited to patents, patent applications, articles, books, and treatises are hereby expressly incorporated by reference in their entirety for any purpose.

The use of the word “syringe” is not limited to any industry standard and includes any of a variety of receptacles provided in different shapes and sizes. Any range described herein will be understood to include the endpoints and all values between the endpoints.

As used herein, “tissue processing” can refer to any number of steps or treatments intended to harvest, clean, or process tissue. Such steps can include washing, removal of collagen strands, mechanical agitation or separation, or removal or filtration of waste and wash from harvested tissue.

As used herein, “adipose tissue” refers to adipose tissue obtained by any means including, for example, liposuction and/or tumescent liposuction. In addition, the adipose tissue may be substantially intact or may be altered by, for example, washing with saline or Lactated Ringer's solution; incorporating antimicrobials, detergents, or other agents; adding therapeutic agents such an analgesics and anti-inflammatories; removing some cells or acellular components; or disrupting or altering the collection process itself including, for example, during liposuction or tumescent liposuction. The adipose tissue can be autologous tissue, allogeneic tissue, or xenogenic tissue (e.g., porcine tissue). Additionally, adipose tissue can refer to particles with multiple adipocyte cells included therein.

Various human and animal tissues can be used to produce products for treating patients. For example, various tissue products have been produced for regeneration, repair, augmentation, reinforcement, and/or treatment of human tissues that have been damaged or lost due to various diseases and/or structural damage (e.g., from trauma, surgery, atrophy, and/or long-term wear and degeneration). Fat grafting, including autologous fat grafting, can be useful for a variety of clinical applications including facial fillers, breast augmentation, buttock augmentation/sculpting, augmentation of other tissue sites, correction of lumpectomy defects, cranial-facial defect correction, and correction of lipoplasty defects (e.g., divots).

To prepare tissue for autologous fat grafting, tissue cleaning and processing is often performed. The process of grafting typically involves steps such as removal of tissue from a patient with a syringe or cannula. The removed tissue is pulled into a tissue processing container where unwanted components of the tissue can be separated and/or the tissue can be cleaned using various solutions. A typical system might include meshes for filtration and separation, cranks connected to mixing blades, and several input and output ports (e.g., to add or remove processing fluids and to transfer tissue). Once the tissue is sufficiently prepared, it must be removed from the container so that some of the tissue may be injected or grafted back into the patient. During transfer steps, vacuum devices help move the tissue from location to location. However, it is desirable to disconnect or change the vacuum pressure configuration during processing steps. In addition, the tissue-carrying tubes that are not in use during any given step should be blocked to maintain the sterility of the system.

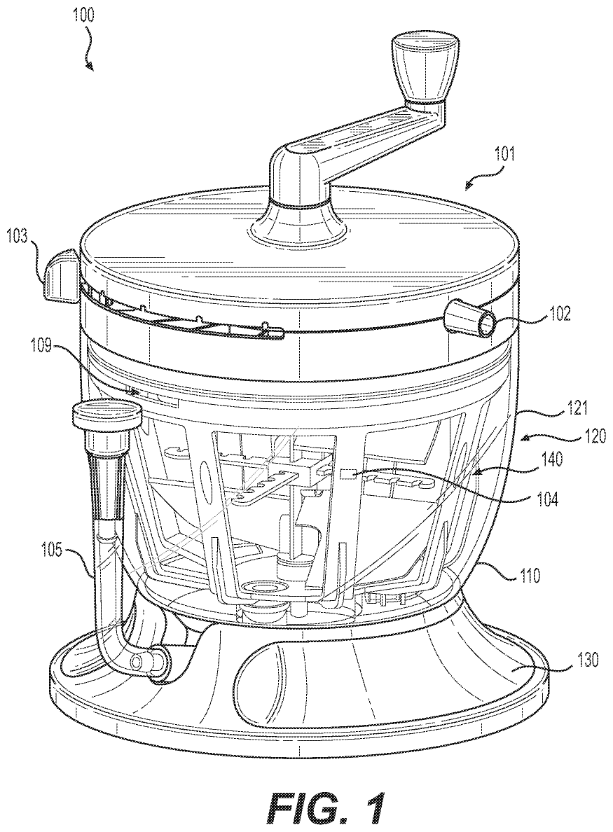

Turning to FIG. 1, an illustrative embodiment of a tissue processing system 100 is shown. As shown, tissue processing system 100 can include container 120 having an exterior wall 121 surrounding an interior volume for holding tissue.

The interior of container 120 can also contain filters, mixing blades, hoses, and other components to enable processing of tissue. Tissue processing system 100 can include flow management system 101 to facilitate operation of the tissue processing system 100. Tissue processing system 100 can further include base 130 to enhance stability of system 100 during use.

Tubes connected to ports 102 provide conduits from the exterior of tissue processing system 100 to the interior through the flow management system 101. Tube restrictor devices within the flow management system 101 can control which tubes are open and which are blocked for a given system configuration. The system configuration is determined by setting multi-position switch 103. In some embodiments, flow management system 101 can hold a blocked tube against at least 1 atmosphere (i.e., about 75 cmHg) of vacuum pressure without leaking.

As used herein, the terms “tube,” “hose,” “conduit,” or similar language will be used interchangeably and will be understood to refer to any passageway having a lumen configured to allow passage or fluids, gases, and/or tissue products therethrough.

In various embodiments, components comprising tissue processing system 100 may be made from a variety of materials suitable for tissue processing. For example, tissue processing system 100 may be manufactured from materials that will enable the device to pass regulatory testing standards, such as ISO 10993-1. Such materials may be sufficiently biocompatible and inert as to not elicit cytotoxic responses during clinical use. Examples of materials potentially suitable for tissue processing system 100 may include plastics, such as polymers (e.g. polyethylene terephthalate (PET), high density polyethylene (HDPE), polyvinyl chloride (PVC), polypropylene (PP), polyimide (TPI), and acrylonitrile butadiene styrene (ABS)), or metals (e.g. stainless steel).

The materials comprising tissue processing system 100 must be able to withstand stresses of device manufacture and sterilization processes, and well as stresses endured during clinical use. For example, the materials of tissue processing system 100 may need to be able to withstand sterilization conditions. Additionally, materials comprising tissue processing system 100 must maintain form and function while exposed to negative pressure generated from suction devices and positive pressure generated from saline or Lactated Ringer's solution positioned above the device and used during the procedure.

Additionally, in certain embodiments, tissue processing system 100 may comprise one or more materials configured to improve operation. For example, exterior wall 110 of tissue processing system 100 may comprise a transparent material so that surgeons or other medical professions may view tissue or other internal components during use. Furthermore, materials of tissue processing system 100 may be optimized for cost efficiency or to simplify the device manufacturing process.

Tissue processing system 100 may be provided in a variety of sizes and configurations. In certain embodiments, tissue processing system 100 may be sized to hold a range of material volumes. For example, tissue processing system 100 may be configured to accommodate input (i.e. harvested) tissues volumes of 40, 50, 60, 70, 80, 90, 100, 120, 140, 160, 180, 200, 250, 300, 350, 400, 450, 500, 600, 700, 800, 900, 1000, 1500, or 2000 ml, or suitable ranges in between. The input tissue volume may be configured to accommodate the volume and rate of tissue input into tissue processing system 100.

In various embodiments, filter structure 140, (i.e. the first chamber of container 120), can comprise maximum harvest level 104, which indicates the maximum recommended level of harvested tissue that should be input into filter structure 140. In various embodiments, maximum harvest level 104 can include a marking or indentation on filter structure 140. In some embodiments, maximum harvest level 104 can include a marking or indentation on container 120, for example, on exterior wall 121. Maximum harvest level 104 can indicate a maximum recommended volume of harvested tissue including, but not limited to, 400, 450, 500, 550, 600, 650, 700, 750, or 800 ml, or suitable ranges in between. In various embodiments, maximum harvest level 104 can indicate a maximum recommended harvested tissue volume of 600 ml. In some embodiments, maximum harvest level 104 can indicate a maximum recommended harvested tissue volume of 700 ml.

In various embodiments, maximum harvest level 104 is configured to allow for sufficient room within container 120 for wash solution, saline, or Ringer's Lactate solution to be input into tissue processing system 100 so that harvested tissue can be adequately washed. For example, in one embodiment, filter structure 140 can accommodate 400 ml of washing solution above maximum harvest line 104, and the second chamber of container 120 can accommodate an additional 200 ml of wash solution. This configuration allows for 600 ml of wash solution to aid in processing harvested tissue within filter structure 140.

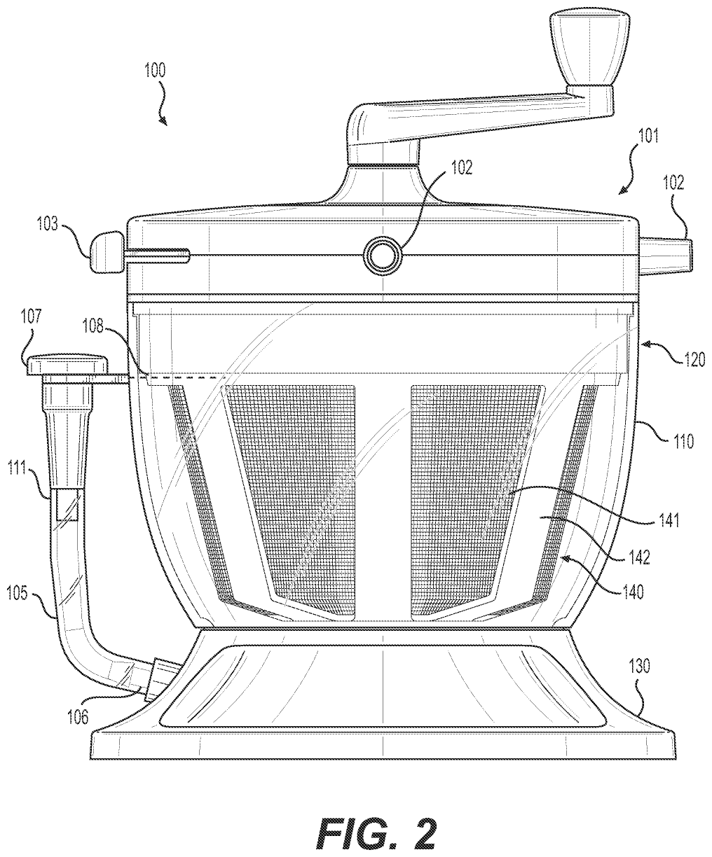

In various embodiments, tissue processing system 100 further comprises opening 109 in the first chamber of container 120 that provides a conduit between the first chamber and the second chamber. In some embodiments, opening 109 is a void in filter structure 140 and is positioned above a maximum fill level of container 120. Opening 109 can aid in preventing a pressure gradient from forming across filter structure 140, specifically, the mesh wall that comprises, in part, filter structure 140, which is illustrated in greater detail in FIG. 2.

During use of tissue processing system 100, a pressure gradient can form if filter structure 140 becomes full and the mesh wall becomes occluded, which may occur during the drain step of the tissue processing procedure when suction is applied to container 120. In this instance, opening 109 can provide a conduit for material to flow from the first chamber to the second chamber of container 120, thus alleviating the pressure gradient and preventing structural failures, such as mesh blow outs.

Referring to FIG. 2, a side-view of tissue processing system 100 from FIG. 1 is shown. Tissue processing system 100 comprises filter structure 140 for processing tissue. Filter structure 140 includes mesh wall 141, which is supported by frame 142. Frame 142 may be provided as a rigid material in various embodiments. In some embodiments, mesh wall 141 divides the interior volume of container 120 into first and second portions or chambers. During autologous fat transfer procedures, lipoaspirate is drawn, usually by means of suction, into tissue processing system 100 through ports 102 when the settings of flow management system 101 are set using multi-position switch 103.

By means of flow management system 101, lipoaspirate and other materials are guided into the first chamber of container 120, which is bounded on its sides by mesh wall 141 and frame 142. Lipoaspirate may comprise fluids, adipose tissue, and waste materials incidentally acquired during the autologous fat transfer procedure. Processing of the lipoaspirate using tissue processing system 100 removes most of the liquids and waste material from the collected lipoaspirate, leaving adipose tissue within the filter structure 140, (i.e. the first chamber of container 120).

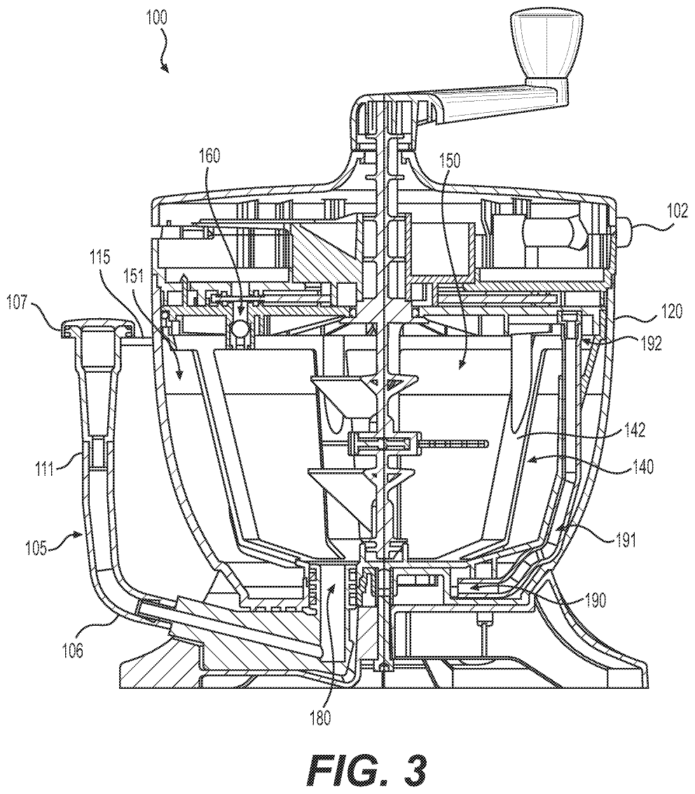

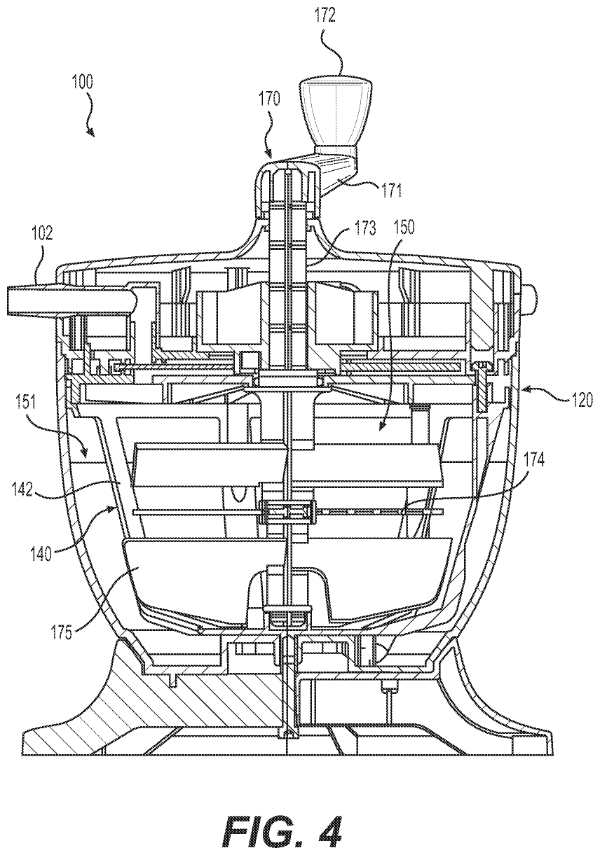

To facilitate processing of lipoaspirate, mesh wall 141 may be configured to allow fluids and small undesired components (e.g., chemicals, blood, non-viable proteins) to pass through its pores while preventing passage of tissue components, such as adipose tissue. Accordingly, during tissue processing, adipose tissue may remain within filter structure 140, while fluids and small undesired components may pass through pores of mesh wall 141, and enter into second chamber formed in the space between the interior wall of container 120 and the exterior of filter structure 140. Diagrams of both chambers of container 120 are illustrated in greater detail in FIGS. 3 and 4.

Referring first to FIG. 3, a first cross-sectional view of tissue processing system 100 is shown. Container 120 includes first chamber 150 and second chamber 151, divided by a diving wall. In various embodiments, the dividing wall is defined, at least in part, by filter structure 140. In various embodiments, the bottom portion of filter structure 140 includes tissue extraction port 180 for removal of materials from within first chamber 150 of container 120.

In various embodiments of the present disclosure, tissue extraction port 180 allows for removal of processed tissue from first chamber 150 at or near the bottom of container 120. The position of tissue extraction port 180 at the bottom of container 120 enables practitioners to remove processed tissue from first chamber 150 without removing the top of tissue processing system 100, without accessing the chamber through a top port, and without manipulating the device from its original position (e.g. picking up or inverting the device). In various embodiments, the processed tissue is drawn into tissue extraction port 180 by means of gravity. In some embodiments, the processed tissue is drawn into tissue extraction port 180 through the application of negative pressure.

In some embodiments, the tissue extraction port 180 can be used to extract fluids, gases, or solids or can be used to insert fluids, gases, or solids. In some embodiments, tissue extraction port 180 is in fluid communication with first chamber 150 of container 120. In other embodiments, tissue extraction port 180 is in fluid communication with second chamber 151 of container 120. Alternatively, tissue processing system 100 can include multiple tissue extraction ports 180 to provide fluid communication to various chambers within tissue processing system 100. In some embodiments, a portion of tissue extraction port 180 can be configured to engage with syringes of various sizes, luer locks, Tuohy Borsts, or any other suitable device.

In various embodiments, tissue processing system 100 further comprises outlet 105 having proximal end 106 and distal end 107. In various embodiments, outlet 105 is in fluid communication with first chamber 150 through tissue extraction port 180. In some embodiments, outlet 105 comprises a flexible tube that maintains an upward position at rest. In some embodiments, outlet 105 can have a sufficient length such that distal end 107 of outlet 105 is positioned above fill level 108, pictured in FIG. 2. In various embodiments, fill level 108 comprises a substantially horizontal marking on exterior wall 121 of container 120. In some embodiments, fill level 108 is horizontally aligned with stop mechanism 160. In some embodiments, stop mechanism 160 prevents users from filling container 120 above a maximum fill level, such as fill level 108. In some embodiments, fill level 108 does not comprise a physical marking, and rather is the maximum volume of material that container 120 can hold.

According to various embodiments of the present disclosure, stop mechanism 160 comprises a valve. In some embodiments, stop mechanism 160 comprises a ball valve, butterfly valve, fill valve, or diaphragm valve. During use of tissue processing system 100, when the fluid and tissue within system 100 reaches the level of fill level 108, stop mechanism 160 is activated and no additional material can enter tissue processing system 100. Thus, in various embodiments, fill level 108 indicates the maximum amount of material (e.g., tissue and fluid) that container 120 can hold.

As recited above, in various embodiments, outlet 105 can have a sufficient length such that distal end 107 of outlet 105 is positioned above fill level 108. Such a configuration can prevent leakage of fluid or tissue from distal end 107 of outlet 105. This may be particularly useful when leaks are most likely, e.g., when a device is being attached to or removed from outlet 105.

Outlet 105 can be provided in a variety of configurations. For example, outlet 105 can include a semi-rigid material that can maintain the shape in which it is molded. For example, in various embodiments, outlet 105 can comprise a metallic or polymeric duct tubing, nylon semi-rigid tubing, or similar materials. In some embodiments, outlet 105 can be provided in a flexible configuration and distal end 107 can be removably connected to system 100 through some connecting means, such as clips or straps.

In various embodiments, outlet 105 can comprise multiple components. In some embodiments, outlet 105 comprises connecting means 111 that can allow for modular connection to various devices, such as in-line fat injection devices or systems. Such devices can facilitate extraction of processed tissue. In some embodiments, a spring actuated manual syringe can be connected to outlet 105 through connecting means 111. Such a syringe can allow for more continuous tissue extraction and eliminate the need for multiple syringes.

In various embodiments, outlet 105 is provided in a flexible configuration. In various embodiments, distal end 107 is positioned above fill level 108 by way of a connecting means. For example, distal end 107 is held in position above fill level 108 by arm 115. Arm 115 can be provided in various embodiments. For example, arm 115 can include a snap-locking feature, a small latched gate, or a Velcro feature to detachably and securely connect distal end 107 to container 120 such that it is positioned above fill level 108. Any means for temporarily and non-destructively affixing distal end 107 to arm 115 such that distal end 107 is positioned above fill level 108 is contemplated by the present disclosure. During use of tissue processing system 100, when a user is ready to extract the processed adipose tissue, they can remove distal end 107 from arm 115 so that they can extract the processed adipose tissue through distal end 107. Additional features of tissue processing system 100 are illustrated in FIG. 4.

FIG. 4 illustrates a second cross-sectional view of tissue processing system 100 from FIG. 1 in a vertical plane normal to the cross-sectional plane from FIG. 3. Tissue processing system 100 comprises mixing system 170, which includes various components used to mix and treat tissue within first chamber 150 of container 120. In various embodiments, mixing system 170 comprises rotatable handle 171 with knob 172. Mixing system 170 further comprises shaft 173, to which at least one mixing blade 175 to facilitate tissue processing, washing, or treatment is attached. In various embodiments of the present disclosure, rotatable handle 171 is operably connected to the at least one mixing blade 175 via shaft 173. In various embodiments, mixing system 170 comprises multiple mixing blades 175.

In various embodiments, mixing system 170 enables a user to mix tissue and fluids within container 120 external to the closed tissue processing system 100, thereby limiting the risk of contamination of the fluids and tissue therein. Users can control the speed and direction of mixing blades 175 by controlling the speed and direction that they use to manipulate rotatable handle 171.

In various embodiments mixing system 170 further comprises protrusions 174 used to facilitate processing, mixing, and treatment of tissue within first chamber 150. Protrusions 174 can be provided with teeth, spikes, or bristles to capture long fibers gathered during acquisition of adipose tissue from the patient's donor site. Long fibers may be undesirable material for reinjection into the body. Thus, protrusions 174 are designed to capture these fibers and prevent them from being removed through tissue extraction port 180 for reinjection into the patient.

In some embodiments, the at least one mixing blade 175 contacts an inner surface of mesh wall 141 during rotation within container 120. During use of system 100, the pores of mesh wall 141 can become clogged by tissue and debris. In the devices of the present disclosure, mixing blade 175 contacts the mesh wall during rotation within container 120, thereby cleaning mesh wall 141. Thus, rotation of mixing blade 175 wipes the pores of mesh wall 141 clear of debris. The pore unclogging feature of devices of the present disclosure facilitates faster filtration during the tissue washing step and allows for rapid introduction of wash solutions into tissue processing device 100, such as saline or Lactated Ringer's solution. The pore unclogging feature of the devices of the present disclosure also reduces the need for users to stop the procedure to unclog the pores of the mesh wall, potentially reducing surgery time.

In various embodiments, tissue processing system 100 is configured such that mixing blades 175 contact mesh wall 141 of filter structure 140, but do not contact frames 142. This feature can be achieved by way of the hexagonal shape of filter structure 140. With the hexagonal (or other polygonal) shape of filter structure 140, the distance between mixing blades 175 and frame 142 is greater than the distance between mixing blades 175 and mesh wall 141. This allows system 100 to be configured such that mixing blades 175 contact and wipe mesh wall 141, but do not contact frames 142 during rotation.

In various embodiments, tissue processing system 100 further comprises a plurality of openings and tubes to facilitate tissue and fluid transfer throughout system 100. For example, referring back to FIG. 3, in various embodiments, waste extraction port 190 is in fluid contact with second chamber 151. During use of system 100, fluids and other waste materials pass through the pores of mesh wall 141 and enter into second chamber 151. To remove this waste product from second chamber 151, waste product is moved through waste extraction port 190, up through waste extraction tubing 191, passes through the appropriate orifice in the flow management system 101, and finally exits container 120 through an external port (not shown). In various embodiments, waste extraction can be facilitated by application of negative pressure through the external port.

In various embodiments, waste extraction tubing 191 is flexible. This allows a tight seal between waste extraction tubing 191 and the various ports to which it connects. In devices of the present disclosure, because waste extraction tubing 191 is flexible, nipple fitting 192 can be used to engage waste extraction tubing 191 near the top of tissue processing system 100. Nipple fitting 192 provides a reliable and long-lasting seal between the two components, limiting the risk of leakage and dislodgement during use of system 100.

In some embodiments, flow management system 101 of tissue processing system 100 comprises a first plate having a plurality of first openings passing therethrough, a second plate having a plurality of second openings passing therethrough, and a third plate having one or more third openings passing therethrough. In various embodiments, the first plate, second plate, and third plate are operably connected, and comprise various tubes, openings, and tube restrictors. Flow management system 101 enables a user to configure the settings of tissue processing system 100 to either collect tissue from the donor site, process collected tissue, or extract either tissue or waste material from system 100, all by adjusting multi-position switch 103 pictured in FIG. 1.

To change the settings within tissue processing system 100, a variety of devices may be implemented. For example, in various embodiments, flow management system 101 may comprise a variety of plates that either open or close a variety of ports and tubes. For example, setting the third plate in a first position places a first subset of the plurality of first openings in fluid communication with a first subset of the plurality of second openings. Setting the third plate in a second position places a second subset of the plurality of first openings in fluid communication with a second subset of the plurality of second openings. Setting the third plate to a third position places a third subset of the plurality of first openings in fluid communication with a third subset of the plurality of second openings. However, multiple systems to open or close various tubes and ports within the system to perform various phases of autologous fat transfer procedure are contemplated within the present disclosure.

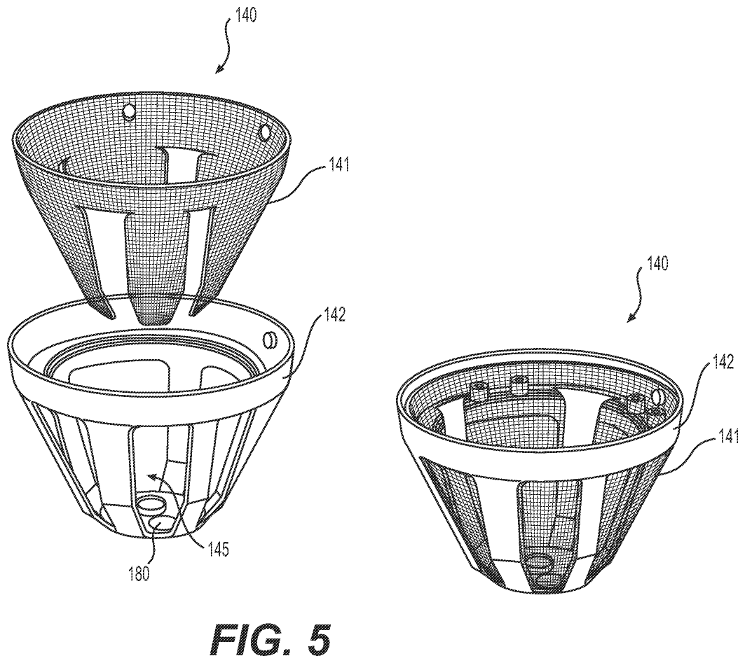

Referring to FIG. 5, both exploded and assembled views of filter structure 140 are shown. In various embodiments, frame 142, supporting mesh wall 141, comprises a rigid material. In various embodiments, frame 142 comprises a rigid material surrounding an upper border of mesh wall 141 and extends along at least a portion of a side wall of mesh wall 141 to a bottom portion of mesh wall 141. In various embodiments, mesh wall 141 may be provided in a variety of forms. For example, mesh wall 141 may be formed from a variety of materials comprising multiple pores.

In some embodiments, mesh wall 141 may be formed from a mesh material such as a porous polymer mesh or metal mesh. In some embodiments, mesh wall 141 may comprise a screen or netting. Mesh wall 141 may be rigid or pliable in various embodiments. The pores of mesh wall 141 may be provided in a variety of sizes suitable for the retention of desired adipose tissue particles. For example, the pores of mesh wall 141 may be about 40, 50, 100, 150, 200, 250, 300, 350, 400, or 500 μm in size. The listed sizes may comprise a single pore size, such as 50 μm, or may be used to define a range of pore sizes, such as 100-150 μm.

In various embodiments, mesh wall 141 comprises a percentage of open area optimized for tissue processing. For example, in some embodiments, the percentage of open area for mesh wall 141 is 20, 25, 30, 35, 40, 45, 50, 55, 60, 65, or 70%. In some embodiments, the percentage of open area for mesh wall 141 is 38-39%.

In various embodiments, frame 142 can be formed of a rigid material, which can provide support and structure to mesh wall 141. In one aspect, frame 142 is formed of liquid-impervious material. In some embodiments, frame 142 can mate with or be formed integral to a top portion of tissue processing system 100. The bottom portion of frame 142 can define, in part, tissue extraction port 180 for removal of materials from within first chamber 150, adjacent to the bottom portion of system 100. In one aspect, frame 142 extends from the top portion of the interior of system 100 to the bottom of the interior of system 100. In some embodiments, frame 142 can surround an upper border of filter structure 140 or mesh wall 141.

In various embodiments, frame 142 can include at least one window 145 defined by frame 142. Various mesh walls 141 can be mated with frame 142 to allow movement of material between first chamber 150 and second chamber 151 of container 120. In various embodiments, mesh wall 141 can be mated within one or more windows 145. In some embodiments, windows 145 extend partially along the vertical length of frame 142. In other embodiments, windows 145 extend completely along the vertical length of frame 142.

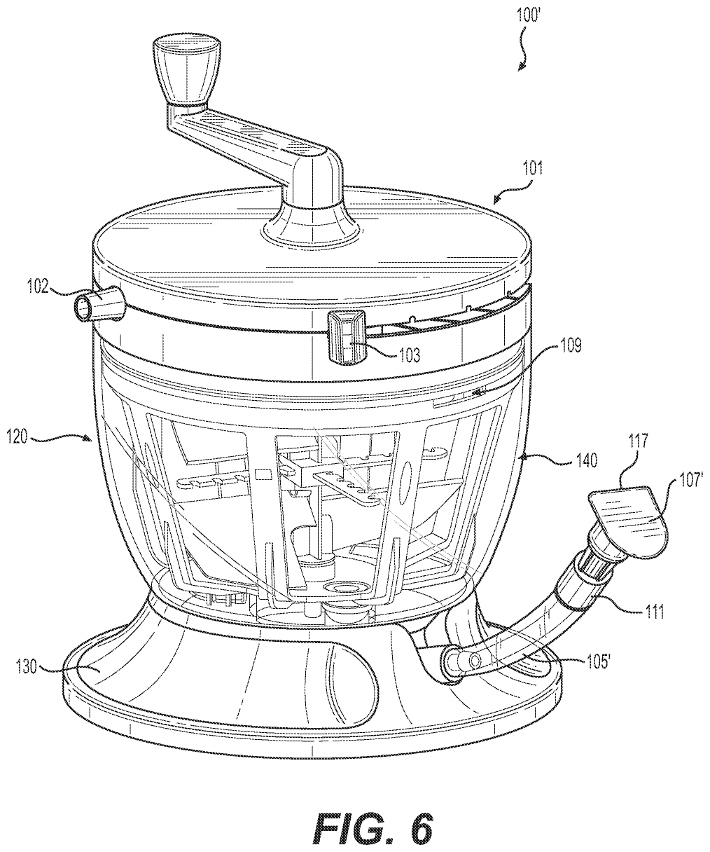

FIG. 6 illustrates a perspective view of an alternative embodiment of tissue processing system 100 from FIG. 1. Tissue processing system 100′ comprises the components of tissue processing system 100, such as flow management system 101, container 120, and base 130, but has a different configuration of outlet 105′. In some embodiments, such as those shown in FIG. 6, outlet 105′ comprises a flexible tube that maintains an upward position at rest. Outlet 105′ comprises distal end 107′. Distal end 107′ comprises various types of access means, such as a flip cap or screw cap that serve as temporary closures of distal end 107′, but can be opened to allow access to second chamber 150. In some embodiments, distal end 107′ comprises a living hinge 117 to allow users to easily open and close distal end 107′.

In some embodiments, distal end 107′ comprises a temporary closure system that is air-tight and liquid-tight. In this configuration, when distal end 107′ is closed, material within container 120 is prevented from flowing up into outlet 105′ as it cannot displace the air therein. Although distal end 107′ does not extend to fill level 108, the vertical configuration of outlet 105′ and the air-tight closure of distal end 107′ prevent leakage from distal end 107′ during use of tissue processing system 100′. Additionally, when distal end 107′ is open, material can then enter into outlet 105′ and users can extract material from container 120.

During use of tissue processing system 100, in various embodiments, lipoaspirate fluid is input by means of suction into system 100 through port 102. In various embodiments, port 102 may be connected to a hose, tube, cannula, or other passageway including a lumen through which fluid, such as lipoaspirate material from an outside source (e.g. in direct contact with the donor site, or separate collection or filtration device), may travel. In various embodiments, such a tube may be provided in a variety of materials, for example, plastic, silicone, nylon or rubber (e.g. Latex). Plastic tubing may comprise polyvinyl carbonate (PVC), a polyolefin, a polyurethane, polyethylene, polypropylene, or a fluoropolymer (e.g. PTFE, FEP, PFA).

Next, lipoaspirate material is washed and treated within first chamber 150 by manipulation of mixing system 170, and optionally, by the addition of various fluids or chemicals, such as saline. Liquids and small molecules pass through the pores of mesh wall 141 into the second chamber 151, and can be extracted from system 100 through waste extraction port 190 and waste extraction tubing 191. Next, either by way of gravity of by the application of negative pressure, processed adipose tissue that remains in first chamber 150 passes through tissue extraction port 180 and into outlet 105 or 105′. Then, in various embodiments, practitioners may remove the processed adipose tissue, for example, by means of distal end 107 or 107′ and prepare it for reinjection into the patient.

To minimize the risk of adipose tissue contamination, tissue processing system 100 may be provided in a sterile state, and may be configured for use in clinical procedures, such as autologous fat facial transfer. This allows surgeons to re-inject processed adipose tissue into the donor site of a patient, without having to go through an added sterilization step.

It will be appreciated, however, that the various steps may be modified, and/or repeated. For example, multiple irrigation and vacuum/cleaning steps may be performed, and additional ports can be included

Additional methods of processing or treatment using the devices described herein are also contemplated and within the scope of the presently claimed inventions.

Other embodiments will be apparent to those skilled in the art from consideration of the specification and practice of this disclosure. It is intended that the specification and examples be considered as exemplary only, with the true scope and spirit of the disclosed devices and methods being indicated by the following claims.

1. A tissue processing system, comprising:
a container, including:
an exterior wall surrounding an interior volume for holding tissue;
a filter structure for processing tissue, the filter structure including a mesh wall;
at least one mixing blade to facilitate tissue washing or treatment, the at least one mixing blade contacting an inner surface of the mesh wall during rotation within the container, thereby cleaning the mesh wall;
a flow management device;
a flexible tube extending from a port located at a bottom portion of the container in fluid communication with an interior portion of the filter structure; and
a stop mechanism to prevent filling of the container above a maximum fill level, wherein the flexible tube has a length sufficient for a distal end of the tube to be positioned above the stop mechanism, thereby preventing leakage of fluid from the container through the flexible tube.

2. The tissue processing system of claim 1, wherein the stop mechanism comprises a valve.

3. The tissue processing system of claim 2, wherein the stop mechanism comprises a ball valve.

4. The tissue processing system of claim 2, wherein the stop mechanism consists of at least one of a butterfly valve, a fill valve, and a diaphragm valve.

5. The tissue processing system of claim 1, wherein the distal end of the flexible tube comprises a flip cap.

6. The tissue processing system of claim 1, wherein the flexible tube is positioned such that the distal end of the flexible tube is positioned above a maximum harvest level.

7. The tissue processing system of claim 1, wherein the container includes a first chamber and a second chamber divided by a dividing wall.

8. The tissue processing system of claim 7, further comprising an opening in the first chamber that provides a conduit between the first chamber and the second chamber.

9. The tissue processing system of claim 7, wherein the dividing wall is defined at least in part by the filter structure.

10. The tissue processing system of claim 1, wherein the filter structure includes a frame supporting an outer surface of the mesh wall.

11. The tissue processing system of claim 10, wherein the frame supporting the mesh wall comprises a rigid material.

12. The tissue processing system of claim 10, wherein the frame comprises a rigid material surrounding an upper border of the mesh wall and the frame extends along at least a portion of a side wall of the mesh wall to a bottom portion of the mesh wall.

13. The tissue processing system of claim 1, further comprising a rotatable handle operably connected to the at least one mixing blade.

14. The tissue processing system of claim 1, further comprising a plurality of openings and tubes to facilitate tissue and fluid transfer throughout the tissue processing system.

15. The tissue processing system of claim 1, wherein the flow management device comprises:
a first plate having a plurality of first openings passing therethrough;
a second plate having a plurality of second openings passing therethrough; and
a third plate having one or more third openings passing therethrough;
wherein the first plate, second plate, and third plate are operably connected and wherein setting the third plate in a first position places a first subset of the plurality of first openings in fluid communication with a first subset of the plurality of second opening, setting the third plate in a second position places a second subset of the plurality of first openings in fluid communication with a second subset of the plurality of second openings, and setting the third plate to a third position places a third subset of the plurality of first openings in fluid communication with a third subset of the plurality of second openings.

16. The tissue processing system of claim 1, wherein the proximal end of the flexible tube is in fluid communication with the interior volume via a port.

17. The tissue processing system of claim 1, wherein the filter structure comprises a hexagonally shaped cross-section.
